(19) United States
(12) Patent Application Publication
(10) Pub. No.: US 2024/0335123 A1
(43) Pub. Date: Oct. 10, 2024

(54) OBTAINING CARDIOVASCULAR AND/OR RESPIRATORY INFORMATION FROM THE MAMMAL BODY

(71) Applicant: Amazec Photonics IP B.V., Oudkarspel (NL)

(72) Inventors: Hendrikus Hubertus Maria Korsten, Eindhoven (NL); Antonius Cornelis Petrus Maria Backx, Veldhoven (NL); Robert Arthur Bouwman, Nuenen (NL); Pieter Lucas Kat, Oudkarspel (NL)

(21) Appl. No.: 18/290,082

(22) PCT Filed: May 10, 2022
(86) PCT No.: PCT/NL2022/050256

§ 371 Date: Nov. 9, 2023

(30) Foreign Application Priority Data
May 11, 2021 (NL) 2028193

(51) Int. Cl. A61B 5/0205

(52) U.S. Cl. CPC A61B 5/02055

(57) ABSTRACT

In the field of obtaining cardiovascular information from the mammal body (2) in a measurement action performed during a measurement period including a period following a moment at which a local spot of a temperature that significantly deviates from the temperature of the mammal body (2) has been created in the blood vascular system of the mammal body (2), values representing temperature difference to a baseline temperature are measured at at least one position close to, on or in the mammal body (2) throughout the measurement period by means of a measurement device (20) including at least one ultra-sensitive sensor (21) with a high resolution that is configured to enable recordation of at least two subsequent indicator dilution curves in a temperature difference course relating to a respective side of the heart. A practical example of the sensor (21) is a photonic sensor such as a Fiber Bragg Grating sensor.

### FIELD OF THE INVENTION

The invention generally relates to methods and systems for obtaining cardiovascular and/or respiratory information from the mammal body.

Among other things, the invention relates to a method for obtaining cardiovascular information from the mammal body, wherein
a measurement action is performed during a measurement period including a period following a moment at which a local spot of a temperature that significantly deviates from the temperature of the mammal body has been created in the blood vascular system of the mammal body,
for the duration of the measurement period and in relation to at least one side of the heart, a temperature difference course is recorded in relation to said local spot in the blood vascular system of the mammal body, which temperature difference course is the overall trend of temperature difference values representing temperature difference to a baseline temperature through time in relation to the respective side of the heart, and
the temperature difference values are measured at at least one measuring position close to, on or in the mammal body by means of a measurement device including at least one sensor.

The invention also relates to a system configured to be used for obtaining cardiovascular information from the mammal body in a measurement action performed during a measurement period including a period following a moment at which a local spot of a temperature that significantly deviates from the temperature of the mammal body has been created in the blood vascular system of the mammal body, comprising:
a measurement device that is configured to measure temperature difference values representing temperature difference to a baseline temperature at at least one position close to, on or in the mammal body throughout the measurement period, and
a processor that is configured to receive the temperature difference values as input from the measurement device, and to record, for the duration of the measurement period and in relation to at least one side of the heart, a temperature difference course in relation to said local spot in the blood vascular system of the mammal body, which temperature difference is the overall trend of the temperature difference values in relation to the respective side of the heart,
   wherein the measurement device includes at least one sensor that is configured to enable recordation of the temperature difference course.

### BACKGROUND OF THE INVENTION

Cardiovascular information is useful in a context of hemodynamic assessment in patients suffering from a heart disease, undergoing cardiac surgery or trauma, or being monitored in the hospital or at home, for example. A practical example of cardiovascular information is a measure of the heart's effectiveness at circulating blood through the circularly system of the body, which measure is commonly referred to as cardiac output. In particular, cardiac output is the volume of blood ejected by the left ventricle or the right ventricle per minute. When a value of the cardiac output is obtained that is outside of a range of values relating to a normal heart condition, this may be an indication that something is wrong with the heart, vascular system, or blood volume, following from a myocardial infarction or blood loss, for example, and that there is a risk of inadequate tissue perfusion.

A well-known way of determining cardiac output relies on thermodilution techniques which involve intravenous injection of an indicator in the form of a cold or hot quantity of fluid and monitoring a temperature change caused by the quantity of fluid passing an appropriate measurement site. In the process, a catheter such as a flow-directed pulmonary artery catheter, also known as Swan-Ganz catheter, is inserted into a central vein and guided through the right atrium and right ventricle to the pulmonary artery, or a femoral, brachial or radial catheter is inserted into a respective femoral, brachial or radial artery. Monitoring the temperature change as mentioned is done by means of at least one sensor mounted on or in the intravascular catheter that is used. The at least one sensor is normally an electronic temperature sensor such as a thermistor. The measured temperature change is processed to calculate the cardiac output.

Conventionally, in order to obtain/calculate a reliable value of the cardiac output, and also of stroke volume, which is the cardiac output divided by the heart rate, the passing indicator is detected only once. One way of compensating for possible recirculation of the injected indicator involves fitting an exponential decay curve through the descending limb of the indicator dilution curve based on the measured temperature change using the descending limb that is obtained in this way for analysis instead of the measured descending limb. The cardiac output is then derived from the area under the corrected indicator dilution curve. Another way of compensating for possible recirculation of the injected indicator relies on an application of models. In this respect, the so-called Local Density Random Walk (LDRW) interpretation is an example.

Another known way of generating a dilution curve is based on intravenous injection of dyes such as Cardio Green. In that case, cardiovascular information can be obtained by using dye-sensing electronic light absorbing sensors placed in the bloodstream, wherein the measurement of dye concentration is based on changes in optical absorbance of the blood at several wavelengths. In this way, a concentration curve can be developed reflecting the concentration of the indicator over time. The area under the first pass concentration curve is inversely proportional to the cardiac output. Yet another known way of generating a dilution curve is based on intravenous injection of salts such as lithium. In that case, cardiovascular information can be obtained by using salt-sensing electronic sensors placed in the bloodstream.

All of the above-described known ways of generating a dilution curve used to quantify cardiovascular function involve disadvantages, a major disadvantage residing in the fact that extensive instrumentation of the subject (patient or animal) under investigation is required. Other disadvantages are the risk to the subject, such as cardiac-rhythm disturbances, infections, perforation of blood vessels or other local damages to the body, and the fact that specially trained physicians are needed to supervise the procedure and to perform at least some of the actions involved.

Other practical examples of cardiovascular information other than cardiac output are ejection fraction of the left ventricle and the right ventricle, and pulmonary and circulating thermal volume, which are directly related to pulmonary and circulating blood volume. For example, the ejection fraction is an excellent predictor of the severity of a cardiac disease, and an increase of the pulmonary thermal volume can indicate failure of the left side of the heart. Ejection fraction is the percentage of blood that is pumped out during a cardiac cycle. The heart is characterized by two ejection fractions, namely the left ventricle ejection fraction and the right ventricle ejection fraction. The pulmonary thermal volume is the volume of the blood between the right ventricle and the left atrium. The circulating thermal volume is the volume of the blood between the left ventricle and the right atrium. The techniques used to assess the ejection fraction, the pulmonary thermal volume and the circulating thermal volume are complex and expensive. These techniques commonly involve inserting catheters into the bloodstream or the heart. Alternatively, radioactive labelled erythrocytes or machines are applied, particularly machines which cannot be used at the bedside or at home, such as CT or MRI scanners. Ultrasound equipment is also applied in some known cases, but such equipment is not useful to determine circulating thermal volume, to mention one limitation.

### SUMMARY OF THE INVENTION

It is an objective of the invention to provide a way of obtaining cardiovascular information from the mammal body which is less complicated, safer and less stressful to the subject under investigation than the currently known ways, yet very reliable and accurate. In view thereof, the invention provides a method as defined in claim 1, which is a method for obtaining cardiovascular information from the mammal body,

wherein
a measurement action is performed during a measurement period including a period following a moment at which a local spot of a temperature that significantly deviates from the temperature of the mammal body has been created in the blood vascular system of the mammal body,
for the duration of the measurement period and in relation to at least one side of the heart, a temperature difference course is recorded in relation to said local spot in the blood vascular system of the mammal body, which temperature difference course is the overall trend of temperature difference values representing temperature difference to a baseline temperature through time in relation to the respective side of the heart,
the temperature difference values are measured at at least one measuring position close to, on or in the mammal body by means of a measurement device including at least one sensor that is configured to enable recordation of the temperature difference course with at least two subsequent indicator dilution curves resulting from at least two subsequent times that said local spot passes at the at least one measuring position, and
the temperature difference course is recorded at least with said at least two subsequent indicator dilution curves.

Advantageous aspects of the method according to the invention are defined in dependent claims 2-23.

The invention also provides a system as defined in claim 24, which is a system configured to be used for obtaining cardiovascular information from the mammal body in a measurement action performed during a measurement period including a period following a moment at which a local spot of a temperature that significantly deviates from the temperature of the mammal body has been created in the blood vascular system of the mammal body, comprising:
a measurement device that is configured to measure temperature difference values representing temperature difference to a baseline temperature at at least one position close to, on or in the mammal body throughout the measurement period, and
a processor that is configured to receive the temperature difference values as input from the measurement device, and to record, for the duration of the measurement period and in relation to at least one side of the heart, a temperature difference course in relation to said local spot in the blood vascular system of the mammal body, which temperature difference is the overall trend of the temperature difference values in relation to the respective side of the heart,
   wherein
the measurement device includes at least one sensor that is configured to enable recordation of the temperature difference course with at least two subsequent indicator dilution curves resulting from at least two subsequent times that said local spot passes at the at least one measuring position, and
the processor is configured to record the temperature difference course at least with said at least two subsequent indicator dilution curves.

Advantageous aspects of the system according to the invention are defined in dependent claims 25-39.

In a further aspect, the invention provides a method for obtaining respiratory information from the mammal body in a measurement action performed during a measurement period, wherein temperature difference values representing temperature difference to a baseline temperature are measured at at least one position close to, on or in the mammal body for the duration of the measurement period by means of a measurement device including at least one sensor that is configured to detect the temperature difference values with a precision of at least 0.0001 K and a dynamic range of at least 10⁵. Advantageously, the at least one sensor of the measurement device is a photonic sensor such as a Fiber Bragg Grating sensor.

The invention also provides a system that is configured to be used for obtaining respiratory information from the mammal body in a measurement action performed during a measurement period. Basically, such a system comprises a measurement device that is configured to measure temperature difference values representing a temperature difference to a baseline temperature at at least one position close to, on or in the mammal body throughout the measurement period, and that includes the above-mentioned at least one sensor that is configured to detect the temperature difference values with a precision of at least 0.0001 K and a dynamic range of at least 10⁵.

The baseline temperature mentioned in the foregoing is normally the general temperature of the respective mammal body or a temperature directly related to the general temperature of the respective mammal body.

### BRIEF DESCRIPTION OF THE DRAWINGS

The invention will now be explained in greater detail with reference to the figures, in which equal or similar parts are indicated by the same reference signs, and in which:

FIG. 1 diagrammatically shows a system according to an embodiment of the invention, and a human body with which the system is associated for the purpose of obtaining cardiovascular information therefrom,

FIG. 2 diagrammatically shows an assembly of components of the system,

FIG. 3 illustrates how an intake of cold fluid in the human body can be realized,

FIGS. 4 and 5 illustrate practical options in respect of sites on the human body where a sensor of the system may be positioned,

FIG. 6 is a representation of measured values of a temperature difference relative to a baseline temperature against time, obtained by means of a sensor positioned on the wrist of a human test subject;

FIG. 7 is an enlarged representation of a portion of FIG. 6,

FIG. 8 is an enlarged representation of a portion of FIG. 7,

FIG. 9 is a representation of simulated values of a temperature difference relative to a baseline temperature against time, relating to the left ventricle of the heart, wherein intravenous cold bolus injection and a mid-esophageal measurement site in the human body are assumed,

FIG. 10 is an enlarged representation of a portion of FIG. 9,

FIG. 11 is a representation of simulated values of a temperature difference relative to a baseline temperature against time, relating to the left ventricle of the heart, wherein intravenous cold bolus injection and a measurement site on the human body at the position of the skin at the wrist overlying the radial artery are assumed,

FIG. 12 is a representation of simulated values of a temperature difference relative to a baseline temperature against time, relating to the left ventricle of the heart, wherein cold air intake and a mid-esophageal measurement site in the human body are assumed,

FIG. 13 is an enlarged representation of a portion of FIG. 12,

FIG. 14 is a representation of simulated values of a temperature difference relative to a baseline temperature against time, relating to both the left ventricle and the right ventricle of the heart, wherein intravenous cold bolus injection and a mid-esophageal measurement site in the human body are assumed,

FIG. 15 is an enlarged representation of a portion of a temperature difference course shown in FIG. 14 and relating to the left ventricle,

FIG. 16 is an enlarged representation of a portion of FIG. 15,

FIG. 17 is an enlarged representation of a portion of FIG. 11,

FIGS. 18 and 19 are representations of simulated values of a temperature difference relative to a baseline temperature against time, relating to both the left ventricle and the right ventricle of the heart, wherein intravenous cold bolus injection and a mid-esophageal measurement site in the human body are assumed, and wherein FIG. 18 relates to a healthy heart and FIG. 19 relates to failing left and right ventricles,

FIG. 20 is a representation of simulated values of a temperature difference relative to a baseline temperature against time, relating to both the left ventricle and the right ventricle of the heart, wherein cold air intake and a mid-esophageal measurement site in the human body are assumed,

FIG. 21 is a representation of simulated values of a temperature difference relative to a baseline temperature against time, relating to both the left ventricle and the right ventricle of the heart, wherein spontaneous breathing and a mid-esophageal measurement site in the human body are assumed, and

FIG. 22 is a representation of simulated values of a temperature difference relative to a baseline temperature against time, relating to both the left side of the heart, wherein spontaneous breathing and a measurement site on the human body at the position of the skin at the wrist overlying the radial artery are assumed.

In respect of the figures being a representation of either measured or simulated values of a temperature difference relative to a baseline temperature against time, it is noted that the temperature difference values shown along the y-axis are expressed in Kelvin relative to the baseline temperature, and that the time values shown along the x-axis are expressed in seconds relative to the start of the respective measurement period.

### DETAILED DESCRIPTION OF EMBODIMENTS

With reference to FIGS. 1-5, a preferred way of putting the invention to practice in the field of obtaining cardiovascular information from the mammal body and recording a temperature difference course in the process is explained. It is to be noted that this preferred way of putting the invention to practice is one example out of numerous other examples covered by the invention, and that the following description should not be understood so as to be limiting the scope of the invention as supported by the present description and defined in the attached claims for at least a part thereof in any way.

In FIG. 1, a system 1 according to an embodiment of the invention is diagrammatically shown. The system 1 is configured to be used for obtaining cardiovascular information from a mammal body 2, which is a human body in the shown example. The system 1 comprises a base unit 10 accommodating a processor 11 and having a display 12, and a measurement device 20 including a sensor 21. which sensor 21 is connectable to the base unit 10 for providing input to the processor 11. The sensor 21 is a Fiber Bragg Grating sensor comprising a Fiber Bragg Grating integrated in an optical fiber 22 such as a glass fiber, and is intended to be put at at least one position close to, on or in the body 2. In this respect, two options are shown in FIG. 1, namely an option of the sensor 21 being positioned on the skin of the wrist 3 overlying a radial artery and an option of the sensor 21 being positioned in the esophagus 4, at the level of the left atrium of the heart, i.e. at the so-called mid-esophageal position. In the first case, it is practical if the sensor 21 is located in an arrangement that is wearable on the skin. In the latter case, it is practical if the sensor 21 is mounted on or in a probe or tube 23 that is suitable for insertion in the esophagus 4.

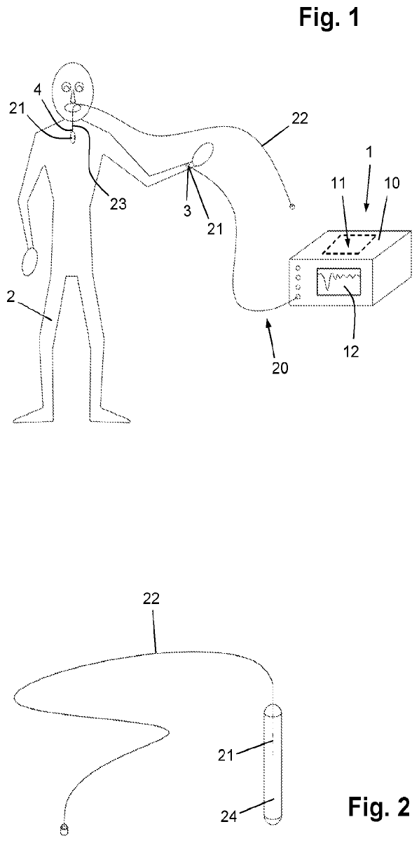

With reference to FIG. 2, it is noted that in the context of the invention, the Fiber Bragg Grating sensor 21 is used as a photonic temperature sensor that is capable of detecting a temperature difference through a wavelength shift as will now be briefly explained. A Fiber Bragg Grating is a small length of optical fiber that comprises a regular pattern of many reflection points that creates a reflection of particular wavelengths of incident light such as laser light. The distance between the reflection points is equal, and the wavelength that matches exactly the distance between two reflection points is reflected by the grating. This reflected wavelength is referred to as the Bragg wavelength. All other wavelengths are transmitted through the grating without being reflected or damped. A Fiber Bragg Grating sensor signal is the narrow spectrum that is reflected at the grating. When a Fiber Bragg Grating is subjected to a change of temperature, the distance of the reflection points changes as a function of thermal expansion of the applied fiber, as a result of which a different wavelength is reflected, i.e. a shift of the Bragg wavelength is obtained.

A Fiber Bragg Grating is not only sensitive to temperature changes, but also to strain. As it is intended to use the Fiber Bragg Grating sensor 21 for detecting a temperature difference, it is practical to accommodate the sensor 21 in a structure 24 that is configured to isolate the sensor 21 from strain and bending, as diagrammatically shown in FIG. 2. Another option is using an additional Fiber Bragg Grating that is constructed in such a way that the thermal expansion coefficient thereof is practically zero, so that the contribution of the strain/bending to the shift of the Bragg wavelength can be determined and excluded.

Other types of sensor for detecting temperature changes could be used, but the use of a Fiber Bragg Grating sensor 21 involves many advantages. To mention one important fact, a Fiber Bragg Grating sensor 21 can be typified as being an ultra-sensitive sensor, as a Fiber Bragg Grating sensor 21 is capable of detecting temperature changes on a milli-Kelvin scale or even a sub-milli-Kelvin scale, over a large dynamic range. Further, Fiber Bragg Grating sensors 21 are known for having a high signal-to-noise ratio, and the response time of this type of sensors is very short due to their very small heat capacity in comparison with the heat capacity of a conventional thermistor, for example.

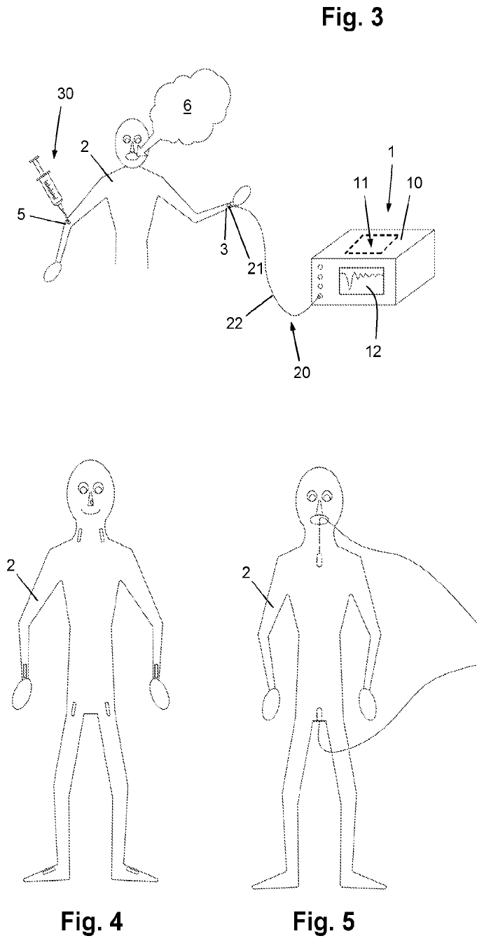

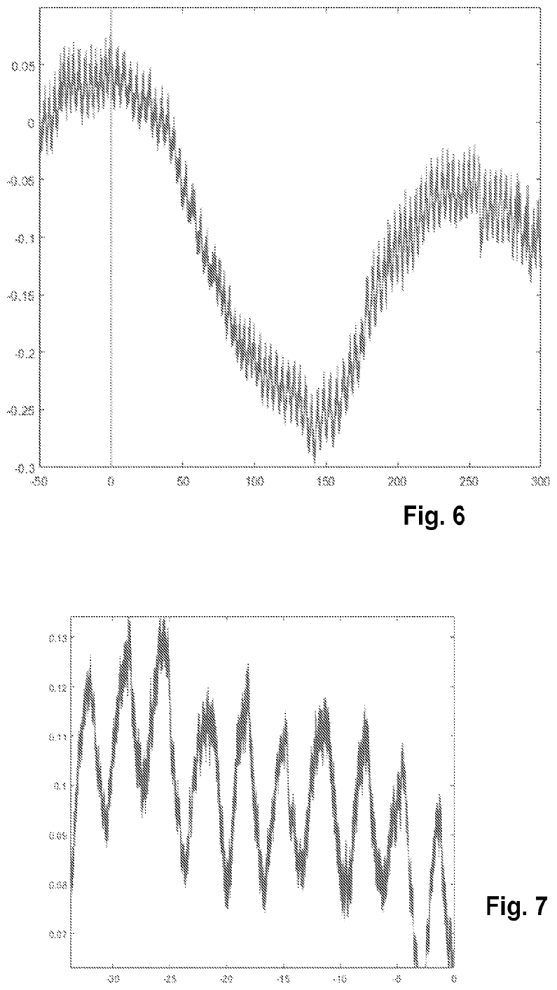

With reference to FIG. 3, it is now explained how the system 1 according to the invention can be used. The sensor 21 is placed on a site of the body 2. This site is preferably outside of the bloodstream, but that does not alter the fact that the invention also covers the use of a sterilized sensor on an intravascular catheter, for example. Other sensors in or outside the body 2 may be used as well and connected to the base unit 10 so as to provide input to the controller 11. As suggested earlier, the sensor 21 may be placed on the skin of the wrist 3 close to the artery, or in the esophagus 4 close to the left atrium. Other sites are also possible, including other sites on the skin above an artery, and a site in the nose, bladder or urethra. In this respect, it is noted that various practical options are illustrated in FIGS. 4 and 5. An advantage of using a mid-esophageal site is that this is a site that is close to the wall of the left atrium and at the same time a site where a stable central temperature of the body 2 is prevailing. Another advantage of using a mid-esophageal site is that this is a site where measurement values relating to both sides of the heart can be obtained with just a single sensor 21.

Further, an intra-venous line 30 is set up and at a certain point a quantity of cold fluid is injected into the bloodstream, at an appropriate position on the body 2, such as at the position of the elbow 5 or the neck. The intra-venous line 30 may be a peripheral or a central intra-venous line. The quantity of fluid is injected as a bolus and the moment of injection is recorded by the processor 11. A practical example of the quantity of cold fluid is 10-30 ml sterile cold saline 0.9% NaCl at a temperature of 0°° C. to 4° C.

At a certain point in time, as a result of the circulation of the blood through the body 2, the injected bolus passes for the first time the site of the body 2 where the sensor 21 is positioned. This is recorded as a first indicator dilution curve, which is related to a first time that a value of a temperature difference to the central body temperature that is taken as a baseline temperature rises and falls, i.e. a first time that a significant temporary deviation from the baseline temperature is found. In view of the fact that the sensor 21 is capable of detecting very small temperature difference values and actually does so, at least one additional indicator dilution curve relating to the respective side of the heart is obtained. This is exactly what is envisaged in the context of the invention, and the measurements are performed during a period of time that covers at least two cycles of blood circulation through the body 2. It is advantageous if during this period, the display 12 is used to depict a course of the measured values in real-time so that a user of the system 1 is enabled to check whether the measurements are performed in the correct way. In view of the fact that dilution of the bolus takes place and the effect of creating a local spot of low temperature is gradually lost as time passes, the repeating, successive indicator dilution curves are recorded with diminishing amplitude. It is an important achievement of the invention that more than one indicator dilution curve in relation to a respective side of the heart is detected after a single cold bolus injection, probably as many as three or four curves, or even more. Each of the first indicator dilution curve and the at least one further indicator dilution curve covers a period of several heartbeats.

It is to be noted that alternatives to the cold bolus injection are feasible. For example, the person who is under investigation may be made to inhale cold/ambient air 6, as diagrammatically depicted in FIG. 3 besides the cold bolus injection option, followed by a period of holding his/her breath or inhaling air at body temperature. The fact is that inhaling air that is cooler than the central body temperature results in minimal fluctuations of the temperature of the blood in the lungs, which drains directly in the left atrium. Contrariwise, an injected bolus has to pass the right side of the heart and the lungs before reaching the left atrium. With the system 1 according to the invention, said minimal fluctuations of the temperature of the blood in the lungs can be continuously measured, thereby functioning as a minimal obtrusive cardiovascular, and also as respiratory monitor at the same time. Another alternative to the cold bolus injection involves creating a local cold spot in the body 2 by placing an object or a substance having a temperature well below the temperature of the body 2 in the mouth. On the other hand, practical options involving creation of a hot pulse instead of a cold pulse are also covered by the invention, such as an option of inhaling air at a temperature that is above body temperature without being inconvenient or even harmful to the person under investigation.

The photonic sensitivity of the sensor 21 to temperature changes is by far exceeding the sensitivity of presently available thermocouples or thermistors, although future technology may probably permit similar results with electronic sensors. It is on the basis of the characteristics of the Fiber Bragg Grating that repeated indicator dilution curves can be obtained after a single injection of the cold indicator. Applying dedicated signal processing, the detected repetition of curves enables accurate determination of one or more cardiovascular parameters. At the same time, there is no need for invasive measurements, which renders the invention very attractive for application in clinical practice on for instance general wards or even at home. In this respect, it is noted that the non-invasive nature of the measurements is enabled on conduction, convection and radiation of the temperature difference that is created through a vessel, the skin or walls of the nose, esophagus and the left atrium, to mention a few practical examples, wherein especially radiation may be denoted as relevant and useful heat transfer factor.

Putting the invention to practice facilitates cardiovascular monitoring and measurement of pulmonary and circulating thermal volumes as well as ejection fractions of the left and right ventricle or perfusion of individual organs such as the prostate. Measurement of pulmonary and circulating thermal volumes can be performed in either an invasive way or a non-invasive way. The option of non-invasively establishing pulmonary and circulating thermal volumes allows for determining these cardiovascular parameters in critically ill patients. In view thereof, the invention may improve treatment and outcome of such patients. Further, the options as mentioned allow for determining the cardiovascular parameters during major (cardiovascular) surgery or in the catherization laboratory to optimize settings for pacemakers and improve the results of minimal-invasive cardiac procedures such as percutaneous or transapical mitral valve repair, closing of septum defects or correction of congenital cardiac defects in babies or young infants.

Conventionally, measurement of pulmonary and circulating thermal volumes by means of thermodilution techniques is not possible, because the recirculation of the cold indicator cannot be measured and only one indicator dilution curve is obtained, which relates to the right side of the heart in the case of a Swan-Ganz catheter. Also in the case of so-called trans-pulmonary thermodilution (PiCCO technique), only one indicator dilution curve is obtained. The fact is that when hitherto known thermodilution techniques are applied, there is no practical way in which the ejection fraction of the left ventricle can be determined directly by means of thermodilution techniques. The estimation of the left ventricular function is usually based on techniques involving X-rays, MRI or ultrasound, or on assumptions and calculations. The present invention allows for making the quantification as desired at the bed-side in a (semi-) continuous way using ultra-sensitive temperature sensors, which may be photonic sensors, yielding useful results. Perfusion of the prostate can be measured with a photonic sensor inserted through the urethra and located at the level of the prostate. It may be useful to assess whether local changes can be found as such local changes can be indicators of (developing) cancer. The sensor may also be used to quantify cardiovascular and/or respiratory information in this context, for instance during surgery with patients having bladder catheterization with a photonic-equipped bladder catheter. Further examples of organs that can be investigated by means of the sensor include the liver and the brain.

The invention also provides a way of using the subtle changes in pulmonary capillary blood temperature in alveolar gas-temperature during inspiration and expiration. The minimal changes in capillary and hence venous pulmonary blood temperature can be picked up by a very fast and highly sensitive and precise (photonic) temperature sensor positioned against the wall of the left atrium from within the esophagus. Cardiovascular parameters such as cardiac output and pulmonary and circulating thermal volumes as well as respiratory parameters such as Presence, Frequency and Volume can be monitored and analyzed non-invasively and continuously once the ultra-sensitive (photonic) temperature sensor is positioned at the correct level in the esophagus, in the vicinity of the left atrium. For the sake of clarity, it is noted that in the present context, the term “non-invasively” is meant to indicate that there is no need for insertion of any device into the bloodstream. In that sense, measurements such as measurements on the skin, inside the nose and inside the esophagus are considered to be non-invasive.

In general, conventional electronic temperature sensors are not capable of detecting a second, third, fourth or fifth passing of the cold indicator, so that a second, third, fourth or fifth indicator dilution curve is not recorded. This is due to the fact that the temperature difference values associated with the reappearing temperature waves are at present below the detection limits of the conventional sensors. As explained in the foregoing, ultra-sensitive sensors such as Fiber Bragg Grating sensors are capable to detect temperature variations with a milli-Kelvin resolution, even fractions of milli-Kelvin, with a very high signal-to-noise ratio over a large dynamic range, and this is the reason why the use of such sensors enables detecting more indicator dilution curves than just the first one. Having information on the basis of at least two successive indicator dilution curves resulting from one and the same cold intake allows for more robust determination of cardiac output, and also enables determination of the circulating thermal volume. In respect of the latter, it is noted that measuring a second or even a third re-appearing indicator dilution curve allows for averaging the mean-transit time differences of successive indicator dilution curves. This is not possible when no or only a single re-appearing indicator dilution curve is available. Further, when indicator dilution curves in relation to both sides of the heart are measured, which can be done by applying an ultra-sensitive sensor at a strategic position such as a mid-esophageal position, pulmonary thermal volume can also be determined.

The temperature resolution of the currently available most sensitive electronic temperature sensors is determined by reproducible change of electric properties of the applied materials as a function of the (change of the) temperature. Extensive filtering, amplification, signal processing and noise reduction are needed to achieve high resolution when electronic temperature sensors are used. In contrast, the temperature changes which are measured by means of photonic sensors are directly based on changes in the length of the optical fiber as a function of temperature on an atomic scale. The changes of the length of the fiber are very accurately measured by analysis of the spectrum of the light reflected by the reflection points in the optical fiber. Using frequency and phase analysis, the resolution can even be as small as 10⁻⁶ Kelvin and perhaps even smaller in the future, while the dynamic range can be very large, such as at least 10⁵.

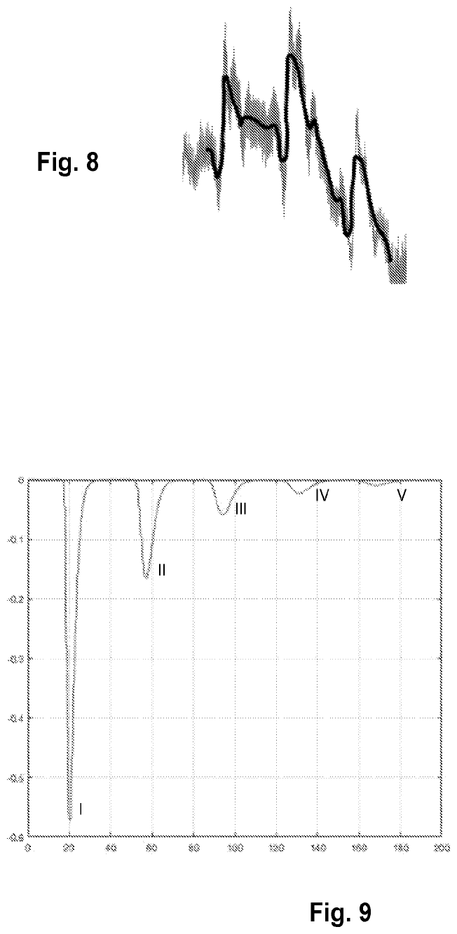

FIG. 6 is a representation of detected temperature difference values expressed in Kelvin against time expressed in seconds, particularly a temperature difference course. The values were obtained during a test in which a 66-year-old healthy male subject received a peripheral injection of 10 ml cold saline in a vein on the back of the hand. The moment of the injection is represented by the vertical line in FIG. 6 and determines the zero value of the time scale. The type of sensor used to detect the values is a Fiber Bragg Grating sensor, and the position of the sensor is on the skin over the radial artery in the wrist at the other side of the person than the side of the injection. The superposed oscillating pattern on the overall trend is obtained as a result of respiratory and cardiac signals. Three important observations are made on the basis of this experiment: 1) it is possible to measure a first indicator dilution curve and a second indicator dilution curve when a Fiber Bragg Grating sensor with a 0.1 mK resolution is positioned on skin overlying radial artery, 2) when zooming in on the temperature difference course, a respiratory signal related to normal breathing at rest (in supine position) can be distinguished, as shown in FIGS. 7, and 3) when zooming in even further, a flow-like signal which may be representative of various phases of the pumping action of the heart can be distinguished within the respiratory signal, on both the descending and ascending portions of the temperature difference course, as shown in FIG. 8. The fact that details as can be seen in FIGS. 7 and 8 are obtained is due to the very high dynamic properties of the measurement system.

In order to explain the measurements, and to estimate the resolution and dynamic range needed for obtaining such measurements, the human circulatory system was modeled in Matlab and Simulink. The model thus obtained can be regarded as the digital twin of the circulation. Both intravenous injection of cold saline and inhalation of cold air or air at room temperature were simulated applying this model. It appears that the actual measurement in the human experiment is confirmed and explained, as will become apparent from the following. The model can provide a basis for more research, and more sophisticated/accurate versions of the model may be developed.

Applying the model that was developed, simulation values are obtained through simulation of a sensor system designed for analysis of the properties of the human circulatory system as a pump system, particularly a sensor system that is capable of monitoring performance of both continuous and pulse pump systems, wherein the latter is applicable to the context of the invention. The following essentials are assumed to be applicable to the monitoring system:
a bolus of liquid with a temperature that differs from the temperature of the pumped liquid or another indicator liquid is injected in the pumped liquid flow. In general, the bolus injection is done fast enough to create a “pulse-shaped” temperature bolus or indicator liquid.
the sensor system accurately and reliably measures the change in temperature, or dilution of indicator liquid with pumped liquid, of the liquid at the outlet side of the pump.
the mechanism for analysis of the pump system characteristics is dilution of the bolus injected with pumped liquid in the pump system and its impact on temperature at the pump outlet.

The principle for detecting pump efficiency is described below.

The injected bolus with volume x [m³] and temperature difference ΔT [K] is assumed to be injected in time interval Δtᵢ. This bolus is injected in the flow φᵢₙ [m3/s] of liquid entering the pump system. As a result, a mixture of pumped liquid and injected bolus enters the pump system. This mixture has the following temperature characteristics:

$$Δ {T}_{m} = ( {φ}_{i n} \cdot {Δt}_{i} + x ) \cdot \frac{Δ T}{x} \qquad ( 1 )$$

ΔTₘ [K] is the temperature of the injected bolus entering the pump system. This bolus as function of time has the same shape as the bolus injected. It is just mixed with pumped liquid and therefore has the average temperature of pumped liquid and injected bolus.

The mixture will enter the pump volume and the pump will dilute this pump volume. Analysis of the dilution enables direct measurement of the pump outlet flow φₒᵤₜ as follows:
The bolus entering the pump has a specific delta-energy ΔE compared to a same volume of the pumped liquid.

$$Δ E = ρ \cdot C \cdot x \cdot Δ T \qquad ( 2 )$$

In this expression ρ [kg/m³] is the density,

$$C [ \frac{J}{kg} \cdot K ]$$

is the specific heat of the liquid
Due to mixture inside the pump volume and corresponding dilution, the temperature bolus is mixed with pumped liquid and the temperature of the pumped liquid at the pump outlet will show temperature variation over a longer time period tₚᵤₗₛₑ [s] than the duration of the pump inlet temperature variation Δtⱼ. Assuming that no energy is lost by the bolus during the stay in the pump and assuming that the pump does not accumulate liquid, so φᵢₙ=φₒᵤₜ, the following holds for the delta energy ΔE:

$$Δ E = ρ \cdot C \cdot {φ}_{out} \cdot {t}_{pulse} \cdot {( Δ T )}_{av} = \frac{ρ \cdot C \cdot {φ}_{out} \cdot {t}_{pulse} \cdot {\int }_{0}^{{t}_{pulse}} Δ {T}_{out} ( t ) dt}{{t}_{pulse}} = ρ \cdot C \cdot {φ}_{out} \cdot {\int }_{0}^{{t}_{pulse}} Δ {T}_{out} ( t ) dt$$

$$Δ E = ρ \cdot C \cdot {φ}_{out} \cdot {\int }_{0}^{{t}_{pulse}} Δ {T}_{out} ( t ) dt = ρ \cdot C \cdot x \cdot Δ T$$

Reworking of this equation gives:

$${φ}_{out} = \frac{x \cdot Δ T}{{\int }_{0}^{{t}_{pulse}} Δ {T}_{out} ( t ) dt} \qquad ( 3 )$$

The accurate measurement of ΔTₒᵤₜ(t) during the whole passage of the temperature pulse at the pump outlet enables accurate calculation of the pump outlet flow

$${φ}_{out} [ \frac{{m}^{3}}{s} ] .$$

Even if the pump is a pulse pump system, the above also holds.

The pump system has an internal volume Vₚᵤₘₚ. The inlet flow φᵢₙ mixes with this volume and the outlet flow is part of the mixed volume. This mixing behavior can be described by:

$$ρ \cdot C \cdot {φ}_{out} \cdot Δ {T}_{out} ( t ) = {\int }_{0}^{∞} {h}_{pump} ( t - τ ) \cdot ρ \cdot C \cdot {φ}_{i n} \cdot {ΔT}_{m} ( τ ) d τ \qquad ( 4 )$$

In this expression hₚᵤₘₚ(t) is the impulse response of the pump system. Assuming the inlet and outlet flow to be constant and equal, so no additional accumulation of liquid occurs in the pump, the equation can be rewritten to:

$$Δ {T}_{out} ( t ) = {\int }_{0}^{∞} {h}_{pump} ( r - τ ) \cdot Δ {T}_{m} ( τ ) d τ \qquad ( 5 )$$

Applying Laplace transform, the equation rewrites to

$$Δ {T}_{out} ( s ) = {H}_{pump} ( s ) \cdot Δ {T}_{m} ( s ) \qquad ( 6 )$$

The pump system can be depicted by its volume Vₚᵤₘₚ to which the inlet flow φᵢₙ is added and of which the outlet flow φₒᵤₜ is subtracted. The following holds with the assumption φᵢₙ=φₒᵤₜ=φ:

$$\frac{d \cdot ρ \cdot C \cdot {V}_{pump} \cdot Δ {T}_{o u t} ( t )}{dt} = ρ \cdot C \cdot {V}_{pump} \cdot \frac{d Δ {T}_{out} ( t )}{dt} = ρ \cdot C \cdot φ \cdot ( Δ {T}_{m} ( t ) - Δ {T}_{out} ( t ) )$$

This results in:

$${V}_{pump} \cdot \frac{d Δ {T}_{out} ( t )}{dt} = φ \cdot ( Δ {T}_{m} ( t ) - Δ {T}_{out} ( t ) ) \qquad ( 7 )$$

$$\frac{d Δ {T}_{out} ( t )}{dt} = \frac{φ}{{V}_{pump}} \cdot ( Δ {T}_{m} ( t ) - Δ {T}_{out} ( t ) )$$

Applying Laplace transform to this equation gives:

$$s \cdot Δ {T}_{out} ( s ) = \frac{φ}{{V}_{pump}} \cdot ( Δ {T}_{m} ( s ) - Δ {T}_{out} ( s ) ) = \frac{1}{{τ}_{pump}} \cdot ( Δ {T}_{m} ( s ) - Δ {T}_{out} ( s ) ) \qquad ( 8 )$$

$$Δ {T}_{out} ( s ) = \frac{1}{{τ}_{pump} \cdot s + 1} \cdot Δ {T}_{m} ( s ) = {H}_{pump} ( s ) \cdot Δ {T}_{𝔪} ( s )$$

In equation (8)

$${τ}_{pump} = \frac{{V}_{pump}}{φ}$$

[s] represents the refreshment time of the pump inner volume Vₚᵤₘₚ. This time constant can be estimated from the recorded pump outlet temperature pulse.

In case a pulse pump system is applied, the observation is made that each outlet flow pulse contains part η of the volume entering the pump system during the pump cycle. In other words, the pump system efficiency can be written as:

$$η = \frac{{V}_{in}}{{V}_{pump}}$$

In case the pulse pump system is described as a discrete time system with sampling time tₛ equal to the pump pulse duration and pump pulse frequency fₚᵤₗₛₑ the following relation applies:

$$Δ {T}_{out} ( i + 1 ) = ( 1 - η ) \cdot Δ {T}_{out} ( i ) + η \cdot Δ {T}_{in} ( i )$$

$${t}_{s} = \frac{1}{{f}_{pulse}}$$

In each pump cycle the volume Vᵢₙ entering and the volume Vₒᵤₜ leaving the pump is a fraction η of the inner pump volume Vₚᵤₘₚ [m³]. Using these characteristics the following holds with i referring to time i·tₛ and (i+1) referring to time (i+1)·tₛ:

giving

$$( z - ( 1 - η ) ) \cdot Δ {T}_{out} ( z ) = η \cdot Δ {T}_{in} ( z ) \qquad ( 9 )$$

$$Δ {T}_{out} ( z ) = \frac{η}{z - ( 1 - η )} \cdot Δ {T}_{in} ( z ) = {H}_{d} ( z ) \cdot Δ {T}_{in} ( z )$$

The discrete time transfer function H_{d}(z) of this pulse pump system enables direct estimation of the efficiency η using equation (9).

Assuming that the pump system is used to pump recycling liquid, as is the case with a cardiovascular system, the volume of the recycling liquid can be directly calculated by calculation of the φₒᵤₜ (equation (3)) and the time between subsequent passes of the temperature pulse. With the time between first passing of the pulse t₁ and second passing of the pulse t₂ the recirculating liquid volume can be calculated by:

$${V}_{recirc} = {φ}_{out} \cdot ( {t}_{2} - {t}_{1} ) \qquad ( 10 )$$

Applying the above to the particular context of a cardiovascular system, the results derived can be used for calculating important parameters of the system.

The cardiac output (CO) can be calculated using equation (3).

$$CO = {φ}_{out} = \frac{x \cdot Δ T}{{\int }_{0}^{{t}_{pulse}} Δ {T}_{out} ( t ) dt} \qquad ( 11 )$$

The circulating thermal volume can be determined using equation (10).

$${v}_{blood} = CO \cdot ( {t}_{2} - {t}_{1} ) \qquad ( 12 )$$

The ratio between volume/heartbeat and volume of the ventricle, i.e. the ejection fraction, can be estimated from the discrete impulse response estimated from the dynamics of the measured temperature pulse that is recorded after the cold bolus injection (cf. equation (9)).

$$Δ {T}_{out} ( z ) = \frac{η \cdot c}{z - ( 1 - η )} \cdot Δ T ( z ) = {H}_{d} ( z ) \cdot Δ T ( z ) \qquad ( 13 )$$

Constant c in this equation represents the mixing of the cold bolus with circulating blood during injection and the heating up of the bolus during circulation in the body. The important parameter to be determined is η (efficiency):

$$η = \frac{{V}_{heartbeat}}{{V}_{ventricle}} \qquad ( 14 )$$

FIG. 9 is a representation of temperature difference values expressed in Kelvin against time expressed in seconds, particularly a temperature difference course, relating to the left ventricle of the heart, obtained through simulation applying the above-mentioned model of the human circulatory system. FIG. 9 is obtained on the basis of input parameters relating to a normal, good functioning heart and to a single intravenous bolus injection of cold saline NaCl 0.9% at a temperature of 4° C., wherein it is assumed that the injection time is 1 second, and wherein it is assumed that the measurements are performed at a mid-esophageal position. All of the shown temperature difference values are within the range as can be detected by a Fiber Bragg Grating sensor, and therefore, the temperature difference course is representative of actual detection results as may be obtained by means of such a sensor.

It can clearly be seen that the temperature difference course includes a number of indicator dilution curves, even as much as five indicator dilution curves I, II, III, IV, V, i.e. four recirculation curves II, III, IV, V following the first curve I. Thus, the detection results actually offer a basis for determining the time difference that is part of equation (10) and that is used in calculating circulating thermal volume. Further, cardiac output and stroke volume can be calculated from the first indicator dilution curve using the well-known equations for doing so. It may be so that in this case, it is assumed that the sensor is positioned outside of the bloodstream, but the equations which have been developed in respect of the well-known use of an intra-vascular catheter are equally applicable.

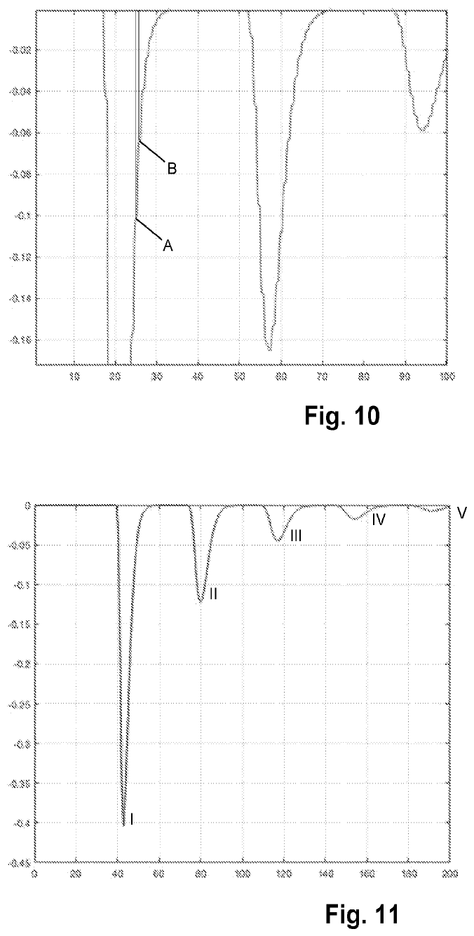

The decrease in temperature as well as the subsequent increase in temperature reflected by the first pass signal offer a basis for calculating the ejection fraction of the left ventricle, wherein useful information can be derived from either one of the descending and ascending limb of the first indicator dilution curve I. In this respect, reference is made to FIG. 10, in which a portion of FIG. 9 is shown in enlarged fashion. It follows from FIG. 10 that small sudden changes of the temperature difference can be distinguished. These small sudden changes are directly related to heartbeats, and this information is used in the process of calculating the ejection fraction, wherein a ratio of the temperature difference values at two subsequent heartbeats is subtracted from 1, assuming that the baseline is really at zero level. For the sake of illustration, positions on the curve of two subsequent heartbeats are indicated in FIG. 10 by A and B. The ejection fraction is

$$E F = 1 - \frac{Δ {T}_{B}}{Δ {T}_{A}}$$

In this expression ΔT_{A} represents the temperature difference value at A, and ΔT_{B} represents the temperature difference at B. Further information about how the ejection fraction is derived from an indicator dilution curve can be found in U.S. Pat. No. 5,383,468, for example.

It follows from an interpretation of FIGS. 9 and 10 that the temperature difference course relates to a good, healthy left ventricle, indeed, because it appears that the cold bolus is transported through the left ventricle in approximately eight heartbeats. As explained, the heartbeats are visible on both the descending and ascending limb of the indicator dilution curve I.

In actual practice, especially when the measurement procedure is repeated one or more times, accurate values of the various cardiovascular parameters can be obtained. In view of the fact that the process of performing the measurement does not need to be bothersome to the subject under investigation, as explained in the foregoing, repeating the measurement procedure can easily be done. The use of cold saline is safe and inexpensive.

The invention also offers the possibility of measuring temperature differences relating to the left atrium, which can also be done by means of an ultra-sensitive sensor such as a Fiber Bragg Grating sensor at the mid-esophageal position. The left atrium is the portion of the heart where an injected bolus arrives first after having passed the lungs. Aspects of the diastolic function of the heart may be monitored in this way, and also an opportunity to detect specific types of malfunctioning of the heart such as atrial fibrillation and other heart conduction disorders is created.

FIG. 11 represents simulation results obtained on the basis of the assumption that the sensor is positioned on the skin at the wrist overlying the radial artery instead of at the mid-esophageal position. In fact, FIG. 11 is comparable to FIG. 9, while a time delay of about 20 seconds is applicable. Thus, FIG. 11 also shows a temperature difference course that includes a number of indicator dilution curves, even as much as five indicator dilution curves I, II, III, IV, V, i.e. four recirculation curves II, III, IV, V following the first curve I.

As suggested in the foregoing, it is also possible to rely on an intake of cold air instead of a cold bolus injection. Although breathing of cold air does not appear to be a “bolus-like” event, as required by standard indicator dilution theories for the purpose of enabling calculation of cardiac output and stroke volume, it may offer a very useful alternative. This is due to the fact that the cold air will be mixed in the lungs and exchange heat quite rapidly with the capillary blood in the lungs. The capillary blood will drain almost immediately into the left atrium, generating an acute decrease in temperature, and this resembles an intravenous bolus after all. In this respect, it is to be noted that after an intravenous injection, the cold blood will not be a real, perfect bolus either once it arrives at the left atrium, since the cold blood had to pass the lungs first, which results in an extended temperature difference course. In fact, it may even be so that similar thermodilution effects are obtained.

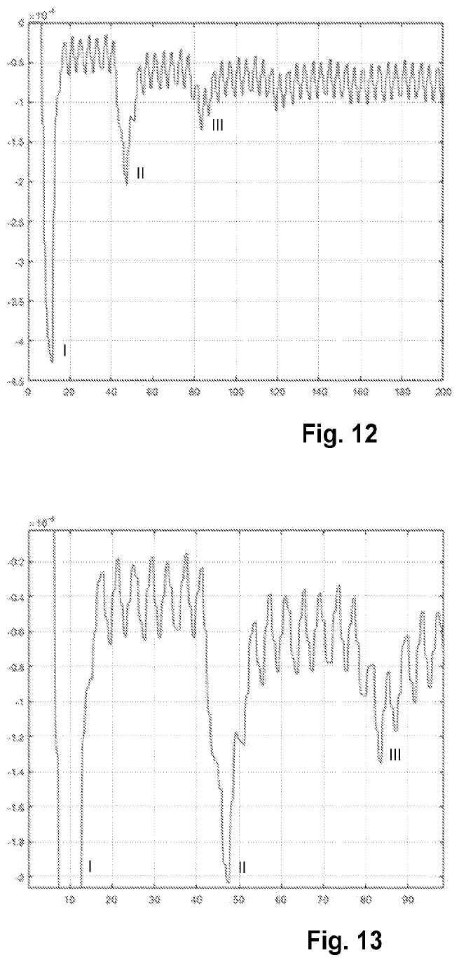

FIG. 12 represents simulation results obtained on the basis of the assumption that the sensor is at the mid-esophageal position and that there has been 1.5 liters cold inhalation of air at −20° C. instead of an intravenous injection of cold fluid. These simulation results relate to the left ventricle and are comparable to the simulation results relating to the injection option. However, the respiratory movement can also be seen in the signal, and after zooming in, also the cardiac signal, due to minimal changes, i.e. increases and decreases, in temperature of the blood due to respiration. A notable fact is that there is almost immediate detection at the left side of the heart of the cold “bolus”, generated by the inhalation of the cold air. FIG. 13 illustrates that after zooming in, the heartbeat can be seen during the passing of the cold indicator, but also during “normal” breathing. Also the ejection fraction of the left ventricle can be measured from the stepwise decrease or increase in temperature difference, as explained earlier, on the basis of the fact that every step signifies a heartbeat.

Apart from cardiovascular aspects, respiratory aspects (Presence, Frequency and Volume) are also important when it comes to patient monitoring and diagnosis in the critically ill. Respiratory monitoring is usually done by analyzing breathing. Practical examples include collecting and analyzing CO₂ in exhaled breath and using sensors such as ECG stickers on the thorax, to name some of the commonly used methods in daily clinical practice. The simulations demonstrate that both respiration and heartbeat can be measured without a need for administering a cold bolus. It appears that the range of the varying temperature of the blood reaching the left atrium is large enough to be measurable at a resolution of 0.1 mK at a position in the esophagus, in the nose, or on the wrist, for example. This means that these parameters can be measured in a test subject, patient or animal in a minimally obtrusive way, and that aspects of both respiration and circulation can be assessed.

In fact, it is possible to apply the invention to only obtain cardiovascular information from the mammal body, to only obtain respiratory information from the mammal body, or to obtain both cardiovascular information and respiratory information from the mammal body. As explained, this is done by applying at least one sensor that is characterized by high resolution and a large dynamic range, which at least one sensor may be a photonic sensor, particularly a Fiber Bragg Grating sensor. Further, as explained, this can be done in a minimal-invasive way, wherein the at least one sensor does not need to be placed in a blood vessel, but may be positioned on the skin overlying an artery, or in the body yet outside of a blood vessel. A mid-esophageal position is an ideal position for performing measurements close to the left atrium. The measurements can be performed after creation of a local cold spot in the blood vascular system of the body, but it is also possible to perform the measurements on the body without such a type of preparatory action.

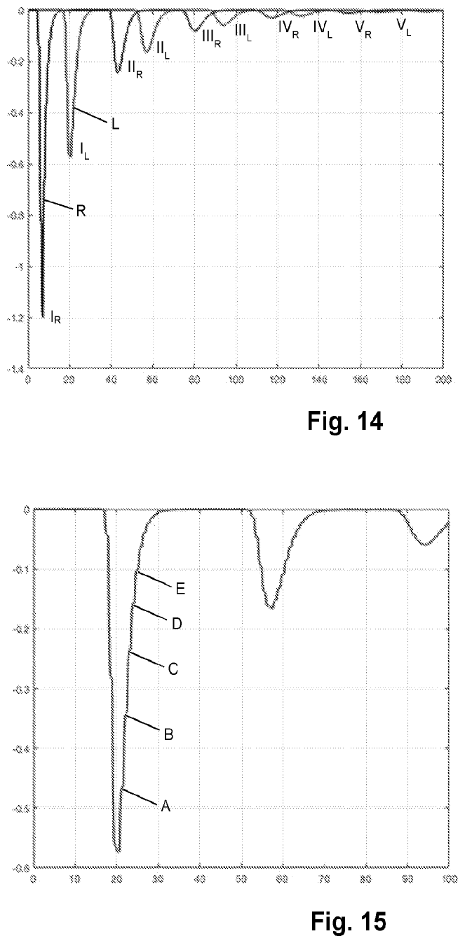

FIG. 14 represents simulation results obtained on the basis of the assumption that the sensor is at the mid-esophageal position and that a 10 ml cold bolus is injected in a peripheral or central vein. The simulation results relate to both the left ventricle and the right ventricle, because at the mid-esophageal position, the ultra-sensitive temperature sensor is not only capable of measuring temperature differences following from the cold bolus passing the left ventricle, but also of measuring temperature differences following from the cold bolus passing the right ventricle. The simulated temperature difference course relating to the left ventricle is indicated as L, and the simulated temperature difference course relating to the right ventricle is indicated as R. The first indicator dilution curve I_{R} is related to the cold bolus arriving at the right atrium for the first time. Further, it can be seen in FIG. 14 that after passing through the lungs, passing the left atrium and the left ventricle, the first indicator dilution curve IL at the left side of the heart is obtained. After passing the entire body, the cold bolus arrives at the right atrium again and the second indicator dilution curve IIR is measured. Subsequently, again after passing the lungs, the second indicator dilution curve II_{L} at the left side of the heart is measured. Depending on the resolution of the sensor, up to five recirculations can be measured, which actually takes place if the resolution is 0.1 mK as can be the case with a Fiber Bragg Grating sensor.

As explained earlier, cardiac output and stroke volume can be calculated from the temperature difference courses. The pulmonary thermal volume can be calculated by multiplying cardiac output by the time differences in mean transit times from the two temperature difference courses L, R, wherein the mean transit times are the times of the indicator dilution curves in the respective temperature difference courses L, R. The following equation is applicable, wherein PTV represents pulmonary thermal volume, CO represents cardiac output and MTT represents mean transit time:

$$P T V = CO \cdot ( ( M T T {I}_{L} ) - ( M T T {I}_{R} ) ) = CO \cdot ( ( M T T {II}_{L} ) - ( M T T {II}_{R} ) ) etc .$$

Also, the circulating thermal volume can be calculated. This is done on the basis of differences between mean transit times in one temperature difference course. The following equation is applicable, wherein CTV represents circulating thermal volume:

$$C T V = CO \cdot ( ( M T T {II}_{R} ) - ( M T T {I}_{R} ) ) = CO \cdot ( ( M T T {II}_{L} ) - ( M T T {I}_{L} ) ) etc .$$

The calculation of the respective volumes is very robust, since more than two recirculations can be considered. The mean transit times and the cardiac output can be calculated from the measured temperature difference courses using an appropriate model known per se, such as the Local Density Random Walk (LDRW) model.

Zooming in, which is possible due to the large dynamic range of the photonic temperature measurements, every individual heartbeat can be seen in the temperature difference course L relating to the left ventricle. FIG. 15 is an enlarged view of a portion of the temperature difference course L relating to the left ventricle as shown in FIG. 14. In FIG. 15, five successive heartbeats A, B, C, D, E are indicated on a descending limb of the course L. Heartbeats are also distinguishable on the ascending limb of the same course L. From the indentations on the course L, ejection fraction, in this case of the left ventricle, can be determined. In a similar manner, namely by zooming in on the temperature difference course R relating to the right ventricle and thereby finding the temperature difference values relating to successive heartbeats, ejection fraction of the right ventricle can be determined.

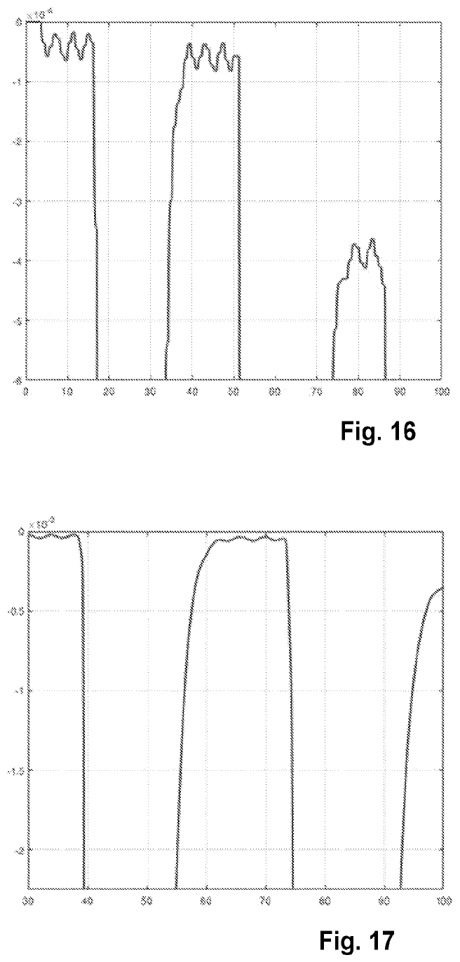

FIG. 16 shows the result of further zooming in on FIG. 15 outside of the part where the indicator dilution curves are depicted. In the direction of the x-axis, the normal breathing pattern can be seen in the signal, and in the normal breathing pattern, the heartbeats can be distinguished. Thus, the simulated results are very well comparable to the results of the actual measurement on a test subject as depicted in FIGS. 6-8.

FIG. 17 shows the result of zooming in on FIG. 11 in which simulation results obtained on the basis of the assumption that the sensor is positioned on the skin at the wrist overlying the radial artery are represented. It can be seen that respiration can also be discerned in these simulation results.

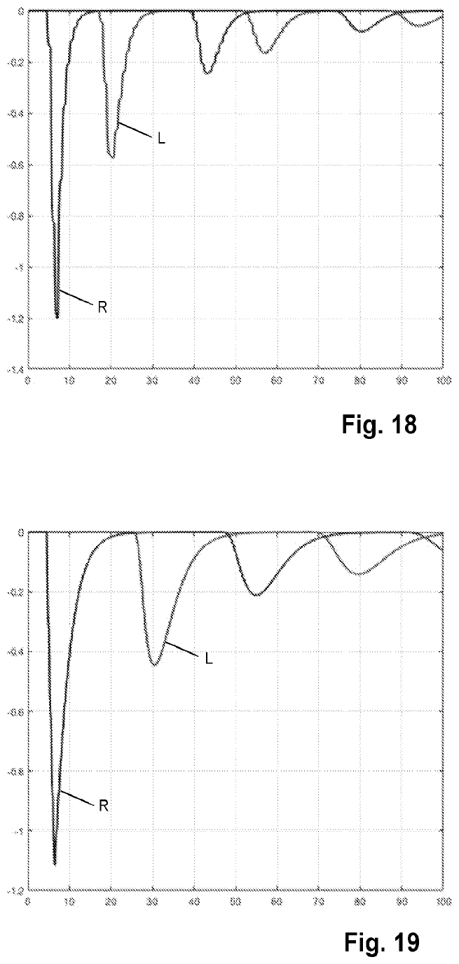

FIG. 18 shows the same simulation results as FIG. 14, but on a different scale. These simulation results relate to a healthy heart. FIG. 19 shows simulation results relating to failing left and right ventricles. From a comparison of FIGS. 18 and 19, it is found that the surface area under the respective temperature difference courses is larger in the case of the failing heart. Also, there is an increase of mean transit times between successive indicator dilution curves in the case of the failing heart, and the circulating thermal volume is higher. Thus, heart failure can be clearly derived from the measurements, by considering one or more aspects of the temperature difference courses.

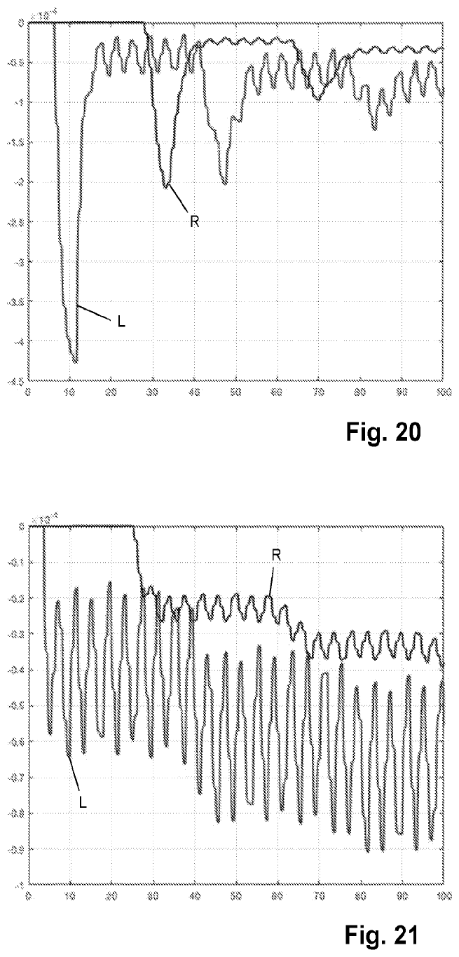

FIG. 20 shows the same temperature difference course as FIG. 12, which relates to the left ventricle in a simulated situation of the sensor being at the mid-esophageal position and 1.5 liters cold inhalation of air at −20° C. Further, FIG. 20 shows the temperature difference course relating to the right ventricle in the same simulated situation. In conformity with FIG. 14, the simulated temperature difference course relating to the left ventricle is indicated as L, and the simulated temperature difference course relating to the right ventricle is indicated as R.

An interpretation of FIG. 20 is as follows. After inhalation of the cold air, the temperature of the capillary blood surrounding the alveoli will—after a short mixing period—almost immediately decrease and enter the left atrium. This explains the short delay that can be seen in the figure. It is to be noted that compared to a situation of cold bolus injection, the first indicator dilution curve that is found relates to the left ventricle. In the situation of cold bolus injection, the cold blood first enters the right atrium, whereas in the situation of the cold air inhalation, the cold blood directly enters the left atrium.

Both the heartbeats and the respiration can be seen on the temperature difference courses shown in FIG. 20. Also, the heart signal within the respiration signal can be discerned, as explained earlier with reference to FIG. 8. Once the cold air is exhaled after a few breaths and in equilibrium with room temperature, the air is usually still colder than body temperature, in view of the fact the room temperature is normally about 20° C. whereas the body temperature is normally about 37.5° C. The measurement results obtained in the situation of cold bolus injection are more useful to calculate cardiac output, but that does not alter the fact that by monitoring the respiratory signal, it is possible to assess the left ventricular systolic, and even more specific diastolic function, which is useful during cardiac-anesthesia, in the coronary care unit or catheterization laboratory, for example. In the case of a failing heart, the indicator dilution curves are higher and are more stretched in the direction of the x-axis. to mention some of the differences which are found in comparison to the case of a healthy heart.

FIG. 21 shows simulated temperature difference courses related to a situation of spontaneous breathing, wherein it is assumed that the sensor is at the mid-esophageal position. With signal-processing techniques, respiratory and cardiac rate can be relatively easily assessed. Thus, application of the invention enables unobtrusive monitoring of the circulation and the respiration, wherein it may even be sufficient to place a sensor on close to a large artery on the skin, such as by means of a patch in the neck above the carotid artery.

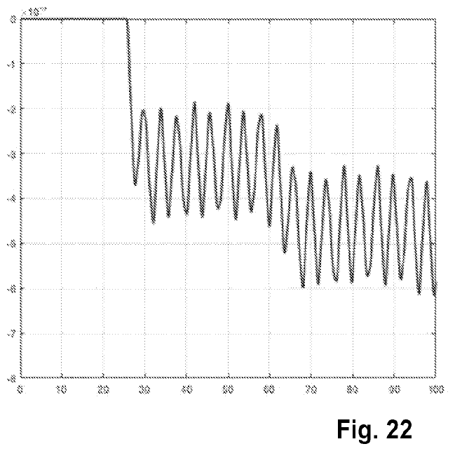

FIG. 22 shows a simulated temperature difference course related to a situation of spontaneous breathing, wherein it is assumed that the sensor is at the wrist, on the skin over the radial artery. Thus, there is only one temperature difference course, relating to the left side of the heart. The cardiac cycle is obscured in this simulation, but with signal processing techniques, the cardiac cycle, particularly rate and rhythm, can be extracted as well. With major arteries close to the heart or in the nose, both signals can be retrieved.

It will be clear to a person skilled in the art that the scope of the invention is not limited to the examples discussed in the foregoing, but that several amendments and modifications thereof are possible without deviating from the scope of the invention as defined in the attached claims. It is intended that the invention be construed as including all such amendments and modifications insofar they come within the scope of the claims or the equivalents thereof. While the invention has been illustrated and described in detail in the figures and the description, such illustration and description are to be considered illustrative or exemplary only, and not restrictive. The invention is not limited to the disclosed embodiments. The drawings are schematic, wherein details which are not required for understanding the invention may have been omitted, and not necessarily to scale.

Notable aspects of the invention are summarized as follows. In the field of obtaining cardiovascular information from the mammal body 2 in a measurement action performed during a measurement period including a period following a moment at which a local spot of a temperature that significantly deviates from the temperature of the mammal body 2 has been created in the blood vascular system of the mammal body 2, a method is provided according to which values representing temperature difference to a baseline temperature are measured at at least one position close to, on or in the mammal body 2 throughout the measurement period by means of a measurement device 20 including at least one ultra-sensitive sensor 21 with a high resolution that is configured to enable recordation of at least two subsequent indicator dilution curves I, II, III, IV, V in a temperature difference course L. R relating to a respective side of the heart. A practical example of the sensor 21 is a photonic sensor such as a Fiber Bragg Grating sensor.

The invention adds to existing diagnostic possibilities as routinely used in hospitals, particularly the possibility to measure not only the cardiac output, but also the circulating thermal volumes in the lungs and body, i.e. the so-called pulmonary thermal volume and the circulating thermal volume, in a minimal-invasive way. That is to say, the sensor(s) does/do not need to be placed in a blood vessel, but may be positioned on the skin overlying an artery (such as a radial, femoral or carotid artery), or in the body yet outside of a blood vessel (such as in the nose or in the esophagus). With a single cold indicator injection or a single intake of cold air by breathing in, both ejection fractions of right and left ventricle can be determined, as well as the cardiac output and the pulmonary thermal volume and the circulating thermal volume in a highly reproducible, transparent and direct way. High resolution measurements of temperature variations are performed in a robust way and there is no need for a complex theoretical/mathematical model including many assumptions and the risk of significant influence of systematic errors. By putting the invention to practice and this enabling direct measurement and non-complex calculation, reliability of the results is very high, all the more so since more than one indicator dilution curve in a temperature difference course is obtained.

1. A method for obtaining cardiovascular information from a mammal body comprising:
performing a measurement action during a measurement period including a period following a moment at which a local spot of a temperature that deviates a deviation amount from the temperature of the mammal body has been created in the blood vascular system of the mammal body
recording, for the duration of the measurement period and in relation to at least one side of the heart, a temperature difference course in relation to the local spot in the blood vascular system of the mammal body, which temperature difference course is an overall trend of temperature difference values representing temperature difference to a baseline temperature through time in relation to the respective side of the heart;
measuring the temperature difference values at at least one measuring position in proximity to, on, or in the mammal body by means of a measurement device including at least one sensor that is configured to enable recordation of the temperature difference course with at least two subsequent indicator dilution curves resulting from at least two subsequent times that the local spot passes at the at least one measuring position; and
recording the temperature difference course at least with the at least two subsequent indicator dilution curves.

2. The method as claimed in claim 1, wherein:
the at least one sensor of the measurement device is configured to enable recordation of the temperature difference course with at least three subsequent indicator dilution curves resulting from at least three subsequent times that the local spot passes at the at least one measuring position; and
the temperature difference course is recorded at least with the at least three subsequent indicator dilution curves.

3. The method as claimed in claim 1, wherein the at least one sensor of the measurement device is configured to detect the temperature difference values with a precision of at least 0.0001 K and a dynamic range of at least 10⁵.

4. The method as claimed in claim 1, wherein the at least one sensor of the measurement device is a photonic sensor.

5. The method as claimed in claim 4, wherein the photonic sensor is a Fiber Bragg Grating sensor.

6. The method as claimed in claim 1, wherein at least one cardiovascular parameter is determined by interpreting a temperature difference course in relation to at least one side of the heart.

7. The method as claimed in claim 6, wherein the at least one cardiovascular parameter is chosen from the group including cardiac output, stroke volume, circulating thermal volume, pulmonary thermal volume, and ejection fraction of a respective ventricle.

8. The method as claimed in claim 7, wherein determining the ejection fraction involves determining a ratio of the temperature difference values at the time of two subsequent heartbeats.

9. The method as claimed in claim 7, wherein determining the circulating thermal volume involves determining a time difference between successive indicator dilution curves in a temperature difference course in relation to a respective side of the heart.

10. The method as claimed in claim 7, wherein:
temperature difference courses are recorded in relation to both sides of the heart; and
determining the pulmonary thermal volume involves determining a time difference between an indicator dilution curve in the one temperature difference course and a subsequent indicator dilution curve in the other temperature difference course.

11. The method as claimed in claim 1, wherein the moment at which a local spot of a temperature that deviates the deviation amount from the temperature of the mammal body has been created in the blood vascular system of the mammal body is a cold intake moment at which a local cold spot has been created in the blood vascular system of the mammal body.

12. The method as claimed in claim 1, wherein the measurement period includes a period directly following a moment at which a local spot of a temperature that deviates the deviation amount from the temperature of the mammal body has been created in the blood vascular system of the mammal body.

13. The method as claimed in claim 12, wherein the measurement period includes a period directly following a moment at which a local spot of a temperature that deviates the deviation amount from the temperature of the mammal body has been created in the blood vascular system of the mammal body by injecting the mammal body intravenously with a volume of a substance having a substance temperature below the temperature of the body.

14. The method as claimed in claim 12, wherein the measurement period includes a period directly following a moment at which a local spot of a temperature that deviates the deviation amount from the temperature of the mammal body has been created in the blood vascular system of the mammal body by placing an object or a substance having a substance temperature below the temperature of the body in the mouth.

15. The method as claimed in claim 12, wherein the measurement period includes a period directly following a moment at which a local spot of a temperature that deviates the deviation amount from the temperature of the mammal body has been created in the blood vascular system of the mammal body by an intake of air having an air temperature below the temperature of the body in the lungs.

16. The method as claimed in claim 12, wherein a duration of the measurement period after the moment at which a local spot of a temperature that deviates the deviation amount from the temperature of the mammal body has been created in the blood vascular system of the mammal body is set so as to cover at least two times an expected blood circulation time through the entirety of the mammal body.

17. The method as claimed in claim 1, wherein the at least one sensor of the measurement device is kept at a position outside of the mammal body throughout the measurement period.

18. The method as claimed in claim 1, wherein the at least one sensor of the measurement device is kept at a position elose in proximity to or on the skin of the mammal body throughout the measurement period.

19. (canceled)

20. The method as claimed in claim 1, wherein the at least one sensor of the measurement device is kept at a position inside the mammal body) throughout the measurement period.

21. The method as claimed in claim 1, wherein the at least one sensor of the measurement device is kept either at a position in a blood vessel or at a position outside of the bloodstream.

22. The method as claimed in claim 21, wherein the position outside of the bloodstream is in a portion of the esophagus that is in proximity to the wall of the left atrium.

23. The method as claimed in claim 20, wherein the at least one sensor of the measurement device is mounted on or in a probe.

24.-39. (canceled)
